(19) United States
(12) Patent Application Publication
(10) Pub. No.: US 2016/0213948 A1
(43) Pub. Date: Jul. 28, 2016

(54) SYSTEM AND METHOD TO CREATE TEST OBJECTS FOR RADIATION THERAPY

(71) Applicant: IMPAC Medical Systems, Inc., Sunnyvale, CA (US)

(72) Inventor: Ryan Renne, Saint Louis, MO (US)

(73) Assignee: IMPAC Medical Systems, Inc.

(21) Appl. No.: 15/003,479

(22) Filed: Jan. 21, 2016

Related U.S. Application Data
(60) Provisional application No. 62/106,241, filed on Jan. 22, 2015.

(51) Int. Cl. A61N 5/10

(52) U.S. Cl. CPC A61N 5/1039

(57) ABSTRACT

System and method are disclosed for creating one or more test objects used for radiation therapy having a processor executing a plurality of computer executable instructions. An exemplary method may include defining a plurality of parameters to create a test object, determining a shape of an object, selecting an image modality, and generating a plurality of images based on the test objects based on object parameters, shape, and image modality. The method may also include creating contours on the plurality of test images, generating a radiation therapy treatment plan, creating a radiation therapy dose, and generating a plurality of Digital Imaging and Communication (DICOM) files that represents one or more radiation therapy objects for a particular test anatomy.

### CROSS-REFERENCE TO RELATED APPLICATION

The present application claims the benefit of priority to U.S. Provisional Application No. 62/106,241, filed Jan. 22, 2015, the entire content of which is expressly incorporated herein by reference.

### TECHNICAL FIELD

The disclosure generally relates to radiation therapy or radiotherapy. More specifically, this disclosure relates to systems and methods for creating test objects for use in developing a radiation therapy treatment plan to be used during radiotherapy.

### BACKGROUND

Radiotherapy is used to treat cancers and other ailments in mammalian (e.g., human and animal) tissue. One such radiotherapy technique is a Gamma Knife, by which a patient is irradiated by a large number of low-intensity gamma rays that converge with high intensity and high precision at a target (e.g., a tumor). In another embodiment, radiotherapy is provided using a linear accelerator, whereby a tumor is irradiated by high-energy particles (e.g., electrons, protons, ions, and the like). The placement and dose of the radiation beam must be accurately controlled to ensure the tumor receives the prescribed radiation, and the placement of the beam should be such as to minimize damage to the surrounding healthy tissue, often called the organ(s) at risk (OARs).

The radiation beam may be shaped to match a shape of the tumor, such as by using a multileaf collimator (e.g., multileaf collimator includes multiple tungsten leaves that may move independently of one another to create customized radiation beam shapes). (Radiation is termed “prescribed” because a physician orders a predefined amount of radiation to the tumor and surrounding organs similar to a prescription for medicine).

Traditionally, for each patient, a radiation therapy treatment plan (“treatment plan”) may be created using an optimization technique based on clinical and dosimetric objectives and constraints (e.g., the maximum, minimum, and mean doses of radiation to the tumor and critical organs). The treatment planning procedure may include using a three-dimensional image of the patient to identify a target region (e.g., the tumor) and to identify critical organs near the tumor. Creation of a treatment plan can be a time consuming process where a planner tries to comply with various treatment objectives or constraints (e.g., dose volume histogram (DVH) objectives), taking into account their individual importance (e.g., weighting) in order to produce a treatment plan which is clinically acceptable. This task can be a time-consuming trial-and-error process that is complicated by the various organs at risk (OARs, because as the number of OARs increases (e.g., up to thirteen for a head-and-neck treatment), so does the complexity of the process. OARs distant from a tumor may be easily spared from radiation, while OARs close to or overlapping a target tumor may be difficult to spare.

Computed Tomography (CT) imaging traditionally serves as the primary source of image data for treatment planning for radiation therapy. CT images offer accurate representation of patient geometry, and CT values can be directly converted to electron densities (e.g., Hounsfield units) for radiation dose calculation. However, using CT causes the patient to be exposed to additional radiation dosage. In addition to CT images, magnetic resonance imaging (MRI) scans can be used in radiation therapy due to their superior soft-tissue contrast, as compared to CT images. MRI is free of ionizing radiation and can be used to capture functional information of the human body, such as tissue metabolism and functionality.

Imaging systems such as computed tomography (CT), fluoroscopy, and magnetic resonance imaging (MRI) may be used to determine the location of and track a target (e.g., an organ, a tumor, and the like). MRI is typically used because it provides excellent soft tissue contract without using ionizing radiation as used by CT. An example of a radiotherapy treatment system integrated with an imaging system may include an MRI-Linac, which may use three-dimensional (3D) images of a target (e.g., a tumor). The MRI apparatus of the MRI-Linac may provide a plurality of images that corresponds to a partial map of hydrogen nuclei in tissues of the patient. The patient images may be acquired in a two-dimensional (2D) plane or in a 3D volume. Because organs and tumors move within a patient's body, fast and accurate 3D localization of the target is important. For instance, a target organ or tumor may move because of various types of motion (e.g., respiratory, cardiac, peristalsis or other types of patient motion).

However, radiation therapy treatment plans have to be tested. Typically, a radiation therapy device (e.g., a linear accelerator, a MRI-linac, etc.) are used to generate images of a phantom. The phantom images are used to create a radiation therapy treatment plan (RTP). The phantom allows one to try various beam angles, radiation dosages, and the like to treat a target tumor. Using a phantom causes the radiation therapy device to be used, which limits the use of the device in treating patients with radiotherapy. A method to create a Digital Imaging and Communication (DICOM) phantom generation tool used for creating test data for treatment planning systems is needed.

### BRIEF DESCRIPTION OF THE DRAWINGS

In the drawings, which are not necessarily drawn to scale, like numerals may describe similar components in different views. Like numerals having letter suffixes or different letter suffixes may represent different instances of similar components. The drawings illustrate generally, by way of example, but not by way of limitation, various embodiments, and together with the description and claims, serve to explain the disclosed embodiments. When appropriate, the same reference numbers are used throughout the drawings to refer to the same or like parts. Such embodiments are demonstrative and not intended to be exhaustive or exclusive embodiments of the present apparatuses, systems, or methods.

FIG. 1 illustrates an exemplary radiotherapy system to localize and track a three-dimensional target for radiation therapy.

FIG. 2 illustrates a radiotherapy device, a Gamma Knife, used in the radiotherapy system of FIG. 1.

FIG. 3 illustrates a radiotherapy device, a linear accelerator, used in the radiotherapy system of FIG. 1.

### DETAIL DESCRIPTION

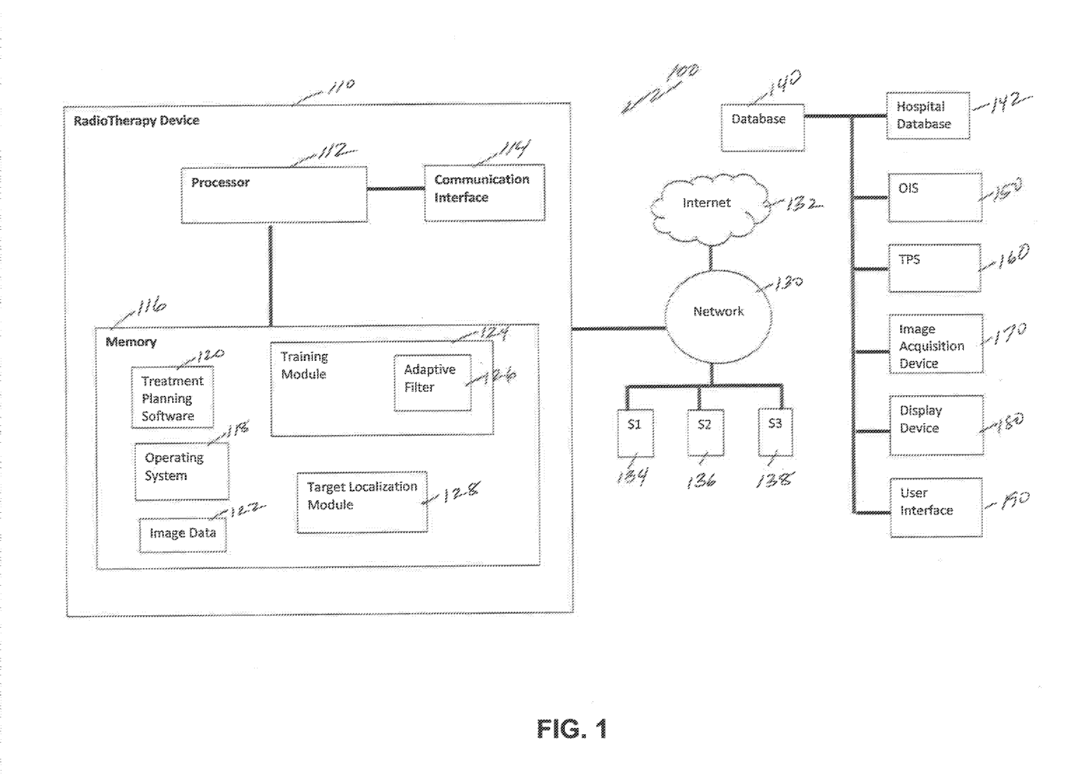

FIG. 1 illustrates an exemplary radiotherapy system 100 for performing target localization and tracking during radiation therapy treatment. Radiotherapy system 100 may include a radiation therapy device 110 connected to a network 120 that is connected to an internet 130. The network 120 can connect the radiation therapy device 110 with a database 140, a hospital database 142, an oncology information system (OIS) 150 (e.g., which may provide patient information), a treatment planning system (TPS) 160 (e.g., for generating radiation therapy treatment plans to be used by the radiotherapy device 110), an image acquisition device 170, a display device 180 and an user interface 190.

The radiotherapy device 110 may include a processor 112, a memory device 116, and a communication interface 114. Memory device 116 may store computer executable instructions, for an operating system 118, treatment planning software 120, a training module 124 that generates an adaptive filter 126, and a target localization module 120 and any other computer executable instructions to be executed by the processor 240.

Processor 112 may be communicatively coupled to the memory device 116, and processor 112 may be configured to execute computer executable instructions stored thereon. For example, processor 112 may execute training module 124 to implement functionalities of both the training module 124 and functionalities of the target localization module 128 in order to determine a location of the target in a patient during administration of radiotherapy. In addition, processor 112 may execute the treatment planning software 120 (e.g., such as Monaco® software manufactured by Elekta) that may interface with training module 124 and target localization module 128.

The processor 112 may be a processing device, include one or more general-purpose processing devices such as a microprocessor, central processing unit (CPU), graphics processing unit (GPU), an accelerated processing unit (APU), or the like. More particularly, processor 112 may be a complex instruction set computing (CISC) microprocessor, reduced instruction set computing (RISC) microprocessor, very long instruction Word (VLIW) microprocessor, a processor implementing other instruction sets, or processors implementing a combination of instruction sets. Processor 112 may also be one or more special-purpose processing devices such as an application specific integrated circuit (ASIC), a field programmable gate array (FPGA), a digital signal processor (DSP), a System on a Chip (SoC), or the like. As would be appreciated by those skilled in the art, in some embodiments, processor 112 may be a special-purpose processor, rather than a general-purpose processor. Processor 112 may include one or more known processing devices, such as a microprocessor from the Pentium™, Core™, Xeon™, or Itanium™ family manufactured by Intel™, the Turion™, Athlon™, Sempron™, Opteron™, FX™, Phenom™ family manufactured by AMD™, or any of various processors manufactured by Sun Microsystems. Processor 112 may also include graphical processing units such as a GPU from the GeForce®, Quadro®, Tesla® family manufactured by Nvidia™, GMA, Iris™ family manufactured by Intel™, or the Radeon™ family manufactured by AMD™. Processor 112 may also include accelerated processing units such as the Desktop A-4(6,8) Series manufactured by AMD™, the Xeon Phi™ family manufactured by Intel™. The disclosed embodiments are not limited to any type of processor(s) otherwise configured to meet the computing demands of identifying, analyzing, maintaining, generating, and/or providing large amounts of imaging data or manipulating such imaging data to localize and track a target or to manipulate any other type of data consistent with the disclosed embodiments. In addition, the term “processor” may include more than one processor, for example, a multi-core design or a plurality of processors each having a multi-core design. Processor 112 can execute sequences of computer program instructions, stored in memory 116, to perform various operations, processes, methods that will be explained in greater detail below.

Memory device 116 can store image data 122 (e.g., 3D MRI, 4D MRI, 2D slices, etc.) received from the image acquisition device 179, or any other type of data/information in any format that the radiotherapy device 110 may use to perform operations consistent with the disclosed embodiments. Memory device 210 may include a read-only memory (ROM), a flash memory, a random access memory (RAM), a dynamic random access memory (DRAM) such as synchronous DRAM (SDRAM) or Rambus DRAM, a static memory (e.g., flash memory, static random access memory), etc., on which computer executable instructions are stored in any format. The computer program instructions can be accessed by the processor 112, read from the ROM, or any other suitable memory location, and loaded into the RAM for execution by the processor 112. For example, memory 116 may store one or more software applications. Software applications stored in memory 116 may include, for example, an operating system 118 for common computer systems as well as for software-controlled devices. Further, memory 116 may store an entire software application or only a part of a software application that is executable by processor 112. For example, memory device 116 may store one or more radiation therapy treatment plans as treatment planning software 120 generated by the treatment planning system 160.

In some embodiments, memory device 116 may include a machine-readable storage medium. While the machine-readable storage medium in an embodiment may be a single medium, the term “machine-readable storage medium” should be taken to include a single medium or multiple media (e.g., a centralized or distributed database, and/or associated caches and servers) that store the one or more sets of computer executable instructions or data. The term “machine-readable storage medium” shall also be taken to include any medium that is capable of storing or encoding a set of instructions for execution by the machine and that cause the machine to perform any one or more of the methodologies of the present disclosure. The term “machine readable storage medium” shall accordingly be taken to include, but not be limited to, solid-state memories, optical and magnetic media.

The radiotherapy device 110 can communicate with a network 130 via a communication interface 114, which is communicatively coupled to processor 112 and memory 116. Communication interface 114 may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a WiFi adaptor), a telecommunication adaptor (e.g., 3G, 4G/LTE and the like), and the like. Communication interface 114 may include one or more digital and/or analog communication devices that permit radiotherapy device 110 to communicate with other machines and devices, such as remotely located components, via a network 130.

The network 130 may provide the functionality of a local area network (LAN), a wireless network, a cloud computing environment (e.g., software as a service, platform as a service, infrastructure as a service, etc.), a client-server, a wide area network (WAN), and the like. Therefore, network 130 can allow data transmission between the radiotherapy device 110 and a number of various other systems and devices, such as for example: the treatment planning system 160, the Oncology Information System 150, and the image acquisition device 170. Further, data generated by the treatment planning system 160, the OIS 150, and the image acquisition device 170 may be stored in the memory 116, database 140, or hospital databased 142. The data may be transmitted/received via network 130, through communication interface 114 in order to be accessed by the processor 112, as required.

In addition, the network 130 may be connected to the internet 132 to communicate with servers or clients that reside remotely and are connected to the internet. As described, network 130 may include other systems S1 (134), S2 (136), S3 (138). Systems S1, S2, and/or S3 may be identical to system 100 or may be different systems. In some embodiments, one or more systems connected to network 130 may form a distributed computing/simulation environment that collaboratively performs image acquisition, target location and target tracking as well other aspects of providing radiotherapy to a patient.

Additionally, radiotherapy system 100 may communicate with the database 140 or the hospital database 142 in order to execute one or more programs stored remotely. By way of example, database 140, hospital database 142, or both may include relational databases such as Oracle™ databases, Sybase™ databases, or others and may include non-relational databases, such as Hadoop sequence files, HBase, Cassandra, or others. Such remote programs may include, for example, oncology information system (OIS) software or treatment planning software. The OIS software, for instance, may be stored on the hospital database 142, the database 140, or the OIS 150. The treatment planning software, for example, may be stored on the database 140, the hospital database 142, the treatment planning system 160 or the OIS 150. Thus, for instance, radiotherapy device 110 may communicate with the hospital database 142 to implement functionalities of the oncology information system 150.

Systems and methods of disclosed embodiments, however, are not limited to separate databases. In one aspect, radiotherapy system 100 may include database 220 or hospital database 230. Alternatively, database 220 and/or hospital database 230 may be located remotely from the radiotherapy system 100. Database 140 and hospital database 142 may include computing components (e.g., database management system, database server, etc.) configured to receive and process requests for data stored in memory devices of database 140 or hospital database 142 and to provide data from database 220 or hospital database(s) 230. One skilled in the art would appreciate that databases 140, 142 may include a plurality of devices located either in a central or distributed manner.

In addition, radiotherapy device 110 may communicate with database 140 through network 130 to send/receive a plurality of various types of data stored on database 140. For example, in some embodiments, database 140 may be configured to store a plurality of images (e.g., 3D MRI, 4D MRI, 2D MRI slice images, CT images, 2D Fluoroscopy images, X-ray images, raw data from MR scans or CT scans, Digital Imaging and Communications in Medicine (DIMCOM) data, etc.) from image acquisition device 140. Database 140 may store data to be used by the target localization module 128, the training module 124, and the treatment planning software 120. The radiation therapy device 110 may receive the imaging data (e.g., 3D MRI images, 4D MRI images) from the database 120 to order to generate a plurality of adaptive filters models, as described below.

Further, the radiotherapy system 100 can include an image acquisition device 170 that can acquire medical images (e.g., Magnetic Resonance Imaging (MRI) images, 3D MRI, 2D streaming MRI, 4D volumetric MRI, Computed Tomography (CT) images, Cone-Beam CT, Positron Emission Tomography (PET) images, functional MRI images (e.g., fMRI, DCE-MRI and diffusion MRI), X-ray images, fluoroscopic image, ultrasound images, radiotherapy portal images, single-photo emission computed tomography (SPECT) images, and the like) of the patient. Image acquisition device 170 may, for example, be an MRI imaging device, a CT imaging device, a PET imaging device, an ultrasound device, a fluoroscopic device, a SPECT imaging device, or other medical imaging device for obtaining one or more medical images of the patient. Images acquired by the imaging acquisition device 170 can be stored within database 140 as either imaging data and/or test data. By way of example, the images acquired by the imaging acquisition device 170 can be also stored by the radiotherapy device 110 in memory 116.

In an embodiment, for example, the image acquisition device 140 may be integrated with the radiotherapy device 110 as a single apparatus (e.g., a MRI device combined with a linear accelerator, also referred to as a “MRI-Linac” or as an integrated MRI device combined with a Gamma Knife). Such a MRI-Linac can be used, for example, to determine a location of a target organ or a target tumor in the patient, such as to direct radiation therapy according to the radiation therapy treatment plan to a predetermined target.

The image acquisition device 170 can be configured to acquire one or more images of the patient's anatomy for a region of interest (e.g., a target organ, a target tumor or both). Each 2D slice can include one or more parameters (e.g., a 2D slice thickness, an orientation, and a location, etc.). The one or more parameters can be adjusted by using the processor 112, to include the target. For instance, selected features of the 2D slice can be manipulated, e.g., by adjusting the gradient or RF waveform properties. For example, the position of the slice can be varied by changing the basic frequency of the RF pulse and maintaining the same gradient strength. Further, the orientation of the slice can be varied, for example, by using a physically different gradient axis (e.g., the selected slice can be orthogonal to the gradient applied). In an example, the image acquisition device 170 (e.g., an MRI or an MRI-Linac) can acquire a 2D slice in any orientation. For example, an orientation of the 2D slice can include a sagittal orientation, a coronal orientation, or an axial orientation. These orientations can correspond to a magnetic field gradient (e.g., Gx, Gy, or Gz, respectively) associated with the MRI or the MRI-Linac. The processor 112 can adjust a parameter, such as the thickness of the 2D slice, to include the target organ or target tumor. In an example, the thickness of the slice can be calculated and adjusted such as by using Equation 1:

$$T = \frac{2 {Δω}_{s}}{γ {G}_{slice}} \qquad ( Eq . 1 )$$

In Equation 1, T can represent the 2D slice thickness, such as can be measured in units of distance (e.g., millimeters). Δωₛ can represent an excitation bandwidth corresponding to a radio frequency pulse applied at a specified frequency (e.g., Hertz, “Hz”). The letter, γ, can represent a constant called the gyromagnetic ratio (e.g., for protons, γ has a value of 267.54 MHz/Tesla). G_{slice} can represent the magnetic field gradient (e.g., measured in Tesla/meters). In an example, 2D slices can be determined from information such as a 3D MRI volume. Such 2D slices can be acquired by the image acquisition device 170 in “real-time” while a patient is undergoing radiation therapy treatment, for example, when using the radiation therapy device 110.

The treatment planning system 160 may generate and store radiation therapy treatment plans for a particular patient to be treated, radiation therapy treatment plans for other patients, as well as other radiotherapy information (e.g., beam angles, dose-histogram-volume information, the number of radiation beams to be used during therapy, the beam angles, the dose per beam, and the like). For example, treatment planning system 160 may provide information about a particular radiation dose to be applied to the patient and other radiotherapy related information (e.g., type of therapy: such as image guided radiation therapy (IGRT), intensity modulated radiation therapy (IMRT), stereotactic radiotherapy; and the like).

Generating the treatment plan may include communicating with the image acquisition device 170 (e.g., a CT device, a MRI device, a PET device, an X-ray device, an ultrasound device, etc.) in order to access images of the patient and to delineate a target, such as a tumor. In some embodiments, the delineation of one or more organs at risk (OARs), such as healthy tissue surrounding the tumor or in close proximity to the tumor may be required. Therefore, segmentation of the OAR may be performed when the OAR is close to the target tumor. In addition, if the target tumor is close to the OAR (e.g., prostate in near proximity to the bladder and rectum), segmentation of the OAR, the treatment planning system 160 may allow study of the dose distribution not only in the target, but also in the OAR.

In order to delineate a target organ or a target tumor from the OAR, medical images, such as MRI images, CT images, PET images, fMRI images, X-ray images, ultrasound images, radiotherapy portal images, SPECT images and the like, of the patient undergoing radiotherapy may be obtained non-invasively by the image acquisition device 170 to reveal the internal structure of a body part. Based on the information from the medical images, a 3D structure may be obtained. In addition, during a treatment planning process, many parameters may be taken into consideration to achieve a balance between efficient treatment of the target tumor (e.g., such that the target tumor receives enough radiation dose for an effective therapy) and low irradiation of the OAR(s) (e.g., the OAR(s) receives as low a radiation dose as possible), the location of the target organ and the target tumor, the location of the OAR, and the movement of the target in relation to the OAR. For example, the 3D structure may be obtained by contouring the target or contouring the OAR within each 2D layer or slice of an MRI or CT image and combining the contour of each 2D layer or slice. The contour may be generated manually (e.g., by a physician, dosimetrist, or health care worker) or automatically (e.g., using a program such as the Atlas-based Autosegmentation software, ABAS®, manufactured by Elekta, AB, Stockholm, Sweden). In certain embodiments, the 3D structure of a target tumor or an OAR may be generated automatically by the treatment planning system 160.

After the target tumor and the OAR(s) have been located and delineated, a dosimetrist, physician or healthcare worker may determine a dose of radiation to be applied to the target tumor and any OAR proximate to the tumor (e.g., left and right parotid, optic nerves, eyes, lens, inner ears, spinal cord, brain stein, and the like). After the radiation dose is determined for each anatomical structure (e.g., target tumor, OAR), a process known as inverse planning may be performed to determine one or more treatment plan parameters, such as volume delineation (e.g., define target volumes, contour sensitive structures), margins around the target tumor and OARs, dose constraints (e.g., full dose to the tumor target and zero dose to any OAR; 95% of dose to PTV, while spinal cord≦45 Gy, brain stem≦55 Gy, and optic structures<54 Gy receive said dosages respectively), beam angle selection, collimator settings, and beam-on times. The result of inverse planning may constitute a radiation therapy treatment plan that may be stored in the treatment planning system 160 or database 140. Some of these treatment parameters may be correlated. For example, tuning one parameter (e.g., weights for different objectives, such as increasing the dose to the target tumor) in an attempt to change the treatment plan may affect at least one other parameter, which in turn may result in the development of a different treatment plan. Thus, the treatment planning system 160 can generate a tailored radiation therapy treatment plan having these parameters in order for the radiotherapy device 110 to provide radiotherapy treatment to the patient.

In addition, the radiotherapy system 100 includes a display device 180 and a user interface 190. The display device 180 may include one or more display screens that display medical images, interface information, treatment planning parameters (e.g., contours, dosages, beam angles, etc.) treatment plans, a target, localizing a target and/or tracking a target, or any related information to the user. The user interface 190 may be a keyboard, a keypad, a touch screen or any type of device that a user may input information to radiotherapy system 100.

In order for the radiation therapy device 110 to localize and track a target tumor in an anatomical region of interest, for example, processor 112 can execute the target localization module 128. The target acquisition module 120 may utilize the adaptive filter model 126 generated by the training module 124, as described later in this disclosure. Further, the adaptive filter model can include particular domain information (e.g., a spatial domain or a frequency domain), location information (e.g., 3D location in a volume, 3D offset information, or 2D displacement information). The target localization module 128 uses at least one adaptive filter model 126 generated by a training module 124, as described below.

Furthermore, any and all components of the radiotherapy system 100, in an embodiment, may be implemented as a virtual machine (e.g., VMWare, Hyper-V, and the like). For instance, a virtual machine can be software that functions as hardware. Therefore, a virtual machine can include at least one or more virtual processors, one or more virtual memories, and one or more virtual communication interfaces that together function as hardware. For example, the OIS 150, the TPS 160, the image acquisition device 170 could be implemented as a virtual machine. Given the processing power, memory, and computational capability available, the entire radiotherapy system could be implemented as a virtual machine.

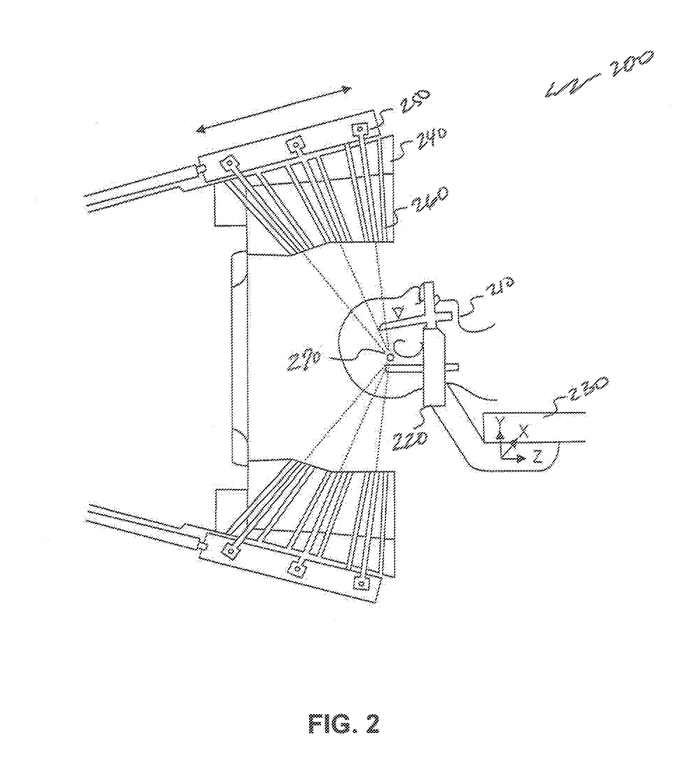

FIG. 2 illustrates an example of one type of radiation therapy treatment device 200, for example, a Leksell Gamma Knife, manufactured by Elekta, AB, Stockholm, Sweden. The Gamma Knife can be configured to utilize the target localization module 128 (shown in FIG. 1), which may remotely access MRI images (e.g., from the image acquisition device 170) to localize a target tumor in the brain. In an embodiment, an MRI apparatus, as an image acquisition device 170, can be integrated with the Gamma Knife. As shown in FIG. 2, during a radiotherapy treatment session, a patient 210 may wear a coordinate frame 220 to keep stable the patient's body part (e.g., the head) undergoing surgery or radiotherapy. Coordinate frame 220 and a patient positioning system 230 may establish a spatial coordinate system, which may be used while imaging a patient or during radiation surgery. Radiotherapy device 200 may include a protective housing 240 to enclose a plurality of radiation sources 250. Radiation sources 250 may generate a plurality of radiation beams (e.g., beamlets) through beam channels 260. The plurality of radiation beams may be configured to focus on an isocenter 270 from different directions. While each individual radiation beam may have a relatively low intensity, isocenter 270 may receive a relatively high level of radiation when multiple doses from different radiation beams accumulate at isocenter 270. In certain embodiments, isocenter 270 may correspond to a target under surgery or treatment, such as a tumor.

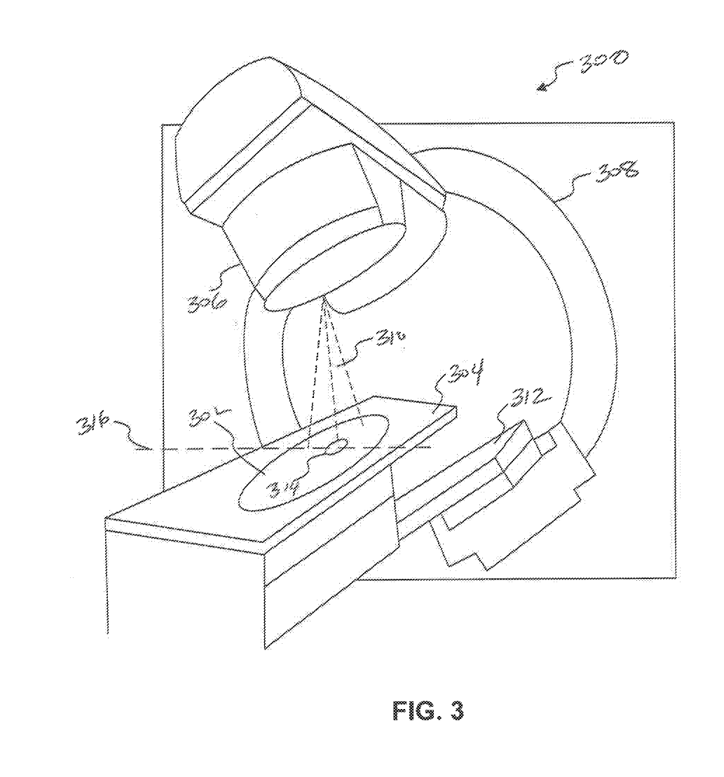

FIG. 3 illustrates another example of a type of radiation therapy device 300 (e.g., a linear accelerator, referred to as a LINAC, manufactured by Elekta, AB, Stockholm, Sweden). Using the linear accelerator 300, a patient 302 may be positioned on a patient table 304 to receive the radiation dose determined by a radiation therapy treatment plan generated by the treatment planning system 160 (shown in FIG. 1). The radiation treatment plan can be used to localize and track a 3D target in a volume, such as a target organ or a target tumor located within the anatomy of the patient 302.

The linear accelerator 300 may include a radiation head 306 connected to a gantry 308 that rotates around the patient 302. The radiation head 306 generates a radiation beam 310 that is directed toward the target organ or target tumor. As the gantry 308 rotates, the radiation head 306 can rotate around the patient 302. While rotating, the radiation head 306 may provide patient 302 with a plurality of varying dosages of radiation depending upon the angle and the shape and size of the tumor according to the treatment plan generated by the treatment planning system 160 (shown in FIG. 1). Because organs and tumors move within a patient's body, fast and accurate 3D localization of the target is important. For instance, a target organ or tumor may move because of various types of motion (e.g., respiratory, cardiac, peristalsis or other types of patient motion). Therefore, the linear accelerator 300 may be configured to localize the target (e.g., organ or tumor) and track the target during radiation therapy treatment by using target localization module 128.

In addition, below the patient table 304, a flat panel scintillator detector 312 may be provided, which may rotate synchronously with the radiation head 306 around an isocenter 314 located on a target organ or a target tumor on the body of the patient 32. The flat panel scintillator can acquire images with the highest achievable signal-to-noise ratio and can be used for verification of the amount of radiation received by the patient 302 during any particular radiation therapy treatment session (e.g., a radiation therapy treatment may require multiple sessions of radiation therapy, where each session is typically referred to as a ‘fraction’). Further, such images are used to determine the geometric accuracy of patient positioning relative to the radiation head 306.

The intersection of an axis 316 with the center of the beam 310, produced by the radiation head 306, is usually referred to as the “isocenter”. The patient table 304 may be motorized so the patient 302 can be positioned with the tumor site at or close to the isocenter 314. For instance, the patient table 304 may change positions relative to one or more other components of the linear accelerator 300, such as to elevate, change the longitudinal position, or the latitudinal position of the patient 302 relative to a therapeutic radiation source located in the radiation head 306.

In an embodiment the linear accelerator 300 may be integrated with the image acquisition device 170 (shown in FIG. 1), such as a magnetic resonance imaging device as a single apparatus (e.g., a MRI-Linac). In such a case, the MRI-Linac may include a “virtual couch” that can “virtually” adjust the alignment of the patient 302 relative to the radiation source when the patient table 304 is configured not to move because of the limited dimensions of a bore size through which the patient table 304 is inserted during radiation therapy treatment. In such an embodiment the MRI-Linac can be used to determine a location of the target and track the target in the patient 302 using the target localization module 128, such as to direct radiation therapy to a predetermined target.

Introduction to CarpeDICOM

CarpeDICOM is a DICOM phantom generation tool used for creating test data for treatment planning systems. It is loosely based on the pre-existing POSDA phantom creation library for Linux. Information regarding POSDA can be found at http://posda.com/Posda/Welcome.html. The series generation portion of code from POSDA was ported to .NET and revamped to support CarpeDICOM's UI. The result is a visual phantom creation engine with added functionality including support for XA Series, RTIONPLAN Series, brachytherapy plans and a host of plan treatment aids.

Default Configurations

Much like POSDA, CarpeDICOM uses a handful of configuration files to populate values for each modality supported. There exists at least one configuration file for each modality. Modalities such as plans have more due to their multiple nested sequences. Any tag represented in a configuration file will be added to the resultant DICOM corresponding to that configuration.

Each DICOM record is constructed by importing all the tags found in the Study.config file then the corresponding series configuration tags. In the case of non-brachytherapy plans, the Beam.config and BeamSegment.config are used to populate beam objects and their control points.

All configuration files are written in XML with the tags grouped by Group number. Certain tags are calculated within CarpeDICOM and are therefore not included in the configurations.

DataSetParameters.config

The DataSetParameters configuration file contains parameters describing the dimensions of the phantom space and its reference frame. Changes made to this file affect all subsequent studies. Image orientation (patient) is defined here as is gantry tilt and the origin of the reference frame.

Preferences

The Preferences dialog found under Edit|Preferences . . . specifies phantom creation parameters. The default write directory is defined here as well as the error logging location. Internal errors of any severity are logged to this file for debugging purposes.

The transfer syntax use when creating phantoms may also be defined here. See section 10 of Part 5: Data Structures and Encoding of the DICOM Standard for more information regarding transfer syntax.

Whether the UIDs are refreshed automatically after a phantom is created can also be specified in this dialog.

Creating DICOM Phantoms

The physical properties of the phantom and its image space are designated in the Create Phantom dialog found under File|New Phantom . . . . Phantoms are constructed as a collection of simple geometric objects, each given an interpreted type, pixel value and dose value. The pixel and dose values defined here are the raw values written to the data and are not to be confused with Hounsfield or Standardized Uptake values which are the result of rescaling.

Phantom parameters can be saved and reloaded within the Create Phantom dialog by selecting File|Save Template. To open a saved template, either drag and drop the template file (*.cdcm) into the Overview pane on in the main window or select File|Open Template in the Create Phantom dialog.

After a geometric object has been added to the list of phantom objects, it can be removed and placed back into the parameter editing panel simply by highlighting the object and clicking “−”.

The parameters in the Spatial Extents section define the imaging area centered on the origin. The phantom objects will be encoded in the pixel data according to which Patient Position and Slice Orientation is selected at create time.

Study

The tags listed in the Study Tag View are for the most part only what are found in the Study.config file. Study date and time are set to the moment when the phantom is created. The Study Instance UID is generated when the study is created. These tags are included in each file that is part of the study and editing one on this page will change it for every file.

Image Series

Tags listed in the Image Series Tag View are shared between all images in the series. Changing a tag here will change its value for each image in the series. However, image data will not be recalculated for changes to these values.

Images

The Image Tag View contains every tag for the selected image that will be written out to file. Editing a tag value here that is shared between images in an image series will change that value for each image containing that tag. Overlapping ROI regions will have the pixel value of the last added ROI in the overlap.

The Image View displays the image data in the Pixel Data tag. The range of displayable pixel values is 0 to 32768, black being 0 and white being 32768.

Note that A GDI+API limitation causes pixel values between 32768 and 65536 to display in CarpeDICOM as 0. Pixel values in this range will still be written out to file correctly.

Note that Image data is created using 16 bits per pixel, regardless of what the Bits Allocated tag is set to.

Transformations and Registration Objects

An image series may be transformed by right-clicking a pre-existing series and selecting “Transform Image Series”. Rotations and translations are preformed in the following order:

Translated Phantom=Translate_{XYZ}(Rotate_{XY}(Rotate_{XZ}(Rotate_{YZ}(Phantom))))

A transformed image series and with a registration object relating the original and transformed series will be produced. The registration object will contain two transformation matrices, the first relating the frame of reference (FOR) of the registration object to the FOR of the original image series. The second matrix relates the FOR of the translated image series to the original series.

X-Ray Angiographic Image Series

Right-clicking a study and selecting “Add XA Series” will bring up the X-Ray Angiograph dialog. Two images are created for each XA series, the first a projected view of the phantom taken from camera vantage point directly above with the top of the image toward the gantry for a Primary Angle of zero. The second image is a projected view 90 degrees greater than the Primary Angle of the first image, the top of the image still towards the gantry. Images are normalized to the range between zero and the value indicated in the Normalize to: field.

Positioner Primary Angle

The Primary Angle represents rotations much like gantry rotations. For positive increments in Primary Angle, the camera rotates clockwise around the Z-axis as would the gantry rotate.

Positioner Secondary Angle

The Secondary Angle represents rotations much like couch pitch rotations. Positive increments in the Secondary Angle rotate the camera in the direction of the gantry around the X-axis.

See section C.8.7.5.1.2 in Part 3: Information Object Definitions of the DICOM Standard for more information regarding Positioner Primary and Positioner Secondary Angles.

Focal Points

If Use Focal Point is selected, images will be calculated using a focal point-based projection algorithm. The focal point will reside a distance of Focal Length from the XA image plane with the phantom in between. The mid-point of the line of focus from the focal point to the center of the XA image plane will always reside at the image space origin.

Structure Sets

The Structure Set View contains the list of Regions of Interest (ROI) that make up the phantom as well as the tags for the structure set. An ROI will exist for each object making up the phantom. ROI details are displayed by selecting an ROI from the list. Contouring is done for each ROI on each image slice using a Marching Squares algorithm.

Plans

General Treatment Aids

Bolus—If the external object of the phantom is a prism, a bolus will be added to the entire study and thus appear in the images, structure set and dose. The bolus will be 50 mm thick and %90 of the length and width of the external prism. If there is no external object or prism, the bolus will not be added to the study but only appear in the plan.

Block—By default, block data is calculated to envelop the open field. The extents of the block will be within the area of the Jaws, the greatest left MLCX leaf position and least right MLCX leaf position.

Compensator—The position is calculated from the number of compensator rows, columns and pixel spacing defined in the Beam.config file. The thickness and transmission default to 50 mm and 0.5, respectively. If the Compensator Mounting Position is set to DOUBLE_SIDED, Source to Compensator Distance data will be calculated and default to 200 mm for each pixel.

Wedge—When a wedge is added to a beam, the option to include the wedge in any control point for that beam becomes available.

Static Vs. Dynamic Beam Types

Static beams are defined as having no movement in the gantry, collimator or couch while the beam is delivering dose. Therefore a control point is needed at the start and end of dose delivery, the first describing the machine state and the second describing the amount of dose delivered. This is why the total number of control points for static beams is always even. Two points together define a segment. Between segments while the machine is not delivering dose, the machine or couch is allowed to move.

Dynamic beams allow movement while the beam is delivering dose. Therefore the total number of control points for dynamic beams does not have to be even.

For static beams, CarpeDICOM adds control points in pairs. By default, the second control point will have a meterset weight of zero.

For dynamic beams, control points are added individually.

Meterset Units (MUs)

MUs assigned to a control point are delivered between that control point and the subsequent control point. The last control point in a beam does not deliver any MUs and is not included in the Final Cumulative Meterset Weight calculation.

Spot Scanning

Spot scanning only applies to RT Ion Plans with a Scan Mode set to Modulated. By default, there are 100 spot scans distributed in a 10×10 square of scans with extents 5 mm inside the jaw positions. Spot Scan Meterset Weights for each scan is the Final Cumulative Meterset Weight divided by the number of spot scans.

Brachytherapy Plans

As defined by the standard, each application setup contains the channel sequence and each channel contains a control point sequence. The RT Brachy Plan details view is laid out with that relationship in mind. Selecting an application setup will display just the channels for that setup. Selecting a channel will display only the control points for that channel. Sources are a separate entity employed by the channels and a sequential relationship does not apply.

The enumerated sources available for use have been modeled on real isotope data. Sources that are primarily gamma-emitting will not have a Source Strength value and those that are primarily beta-emitting will. Additionally, Source Strength Units will be AIR_KERMA_RATE for gamma sources and DOSE_RATE_WATER for beta.

When adding a channel to an application setup, the selected source will be used in that channel.

If the Brachy Treatment Type is set to PDR, pulse-related tags are added to the channel sequence. The Number of Pulses value defaults to 25 and Pulse Repetition Interval to 50.

If Source Movement Type is set to STEPWISE, a channel's Source Applicator Step Size will be added to the channel sequence and default to 5 mm.

Digitally Reconstructed Radiographs

Digitally Reconstructed Radiographs may be created by right-clicking a Plan and selecting “Add RT Image”. The creation parameters are identical to those of X-Ray Angiographs explained above in section 4.5 but only one image is created.

Dose

The Dose Data View displays the dose data for the selected longitudinal position from the Grid Frame Offset Vector. Pixel Rows, Columns and Spacing are the same as those used for the image series pixels. The dose values are the raw values from the Pixel Data before scaling. Pixels are 16 bits regardless of what Bits Allocated is set to.

For overlapping ROIs, the dose in the intersecting region will be dose of the last added ROI.

Reading DICOM

Existing DICOM files may be read in and displayed by either selecting File|Open Patient . . . to read in a directory of DICOM or File|Open File . . . to read a single file; or drag and drop a file or directory into the Overview pane on the left-hand side of the tool.

Note that Explicit Big Endian transfer syntaxes may cannot be read.

Files are displayed in the Overview pane in hierarchical order, highlighting the referential integrity between series for a study.

If a file references another but that file does not exist in the data set, it will show up in red.

If a file has multiple references, the file will be displayed under the first reference and will show up yellow.

If a file has an SOP Instance UID that matches another file in the dataset, both files will show up orange.

The shared attributes for a study and series are displayed in the study and series items, respectively. Attributes that appear in all files within a study or series but have differing values will appear with “<Values Differ>” as the value. Edits made to attributes in a study or series item will be applied to all files within that study or series.

While in ‘Read’ mode, a number of options are available that were not in ‘Create’ mode.

Anonymization

Data may be anonymized by selecting Edit|Anonymize . . . . By default, the anonymization routine will only anonymize Names (VR=PN), Dates and Times (VR=TM, DA), UIDs (VR=UI) and any attribute in the patient demographic group (0x0010). Names and UIDs are randomly generated and Dates and Times are set to the current date/time.

The anonymization dialog allows for additional anonymization by way of the Anonymize.config file. Attributes included in this configuration file will also be randomly anonymized in keeping with the allowable value length for the VR of the tag.

The “Remove Private Attributes” option is selected by default and will remove attributes that are not found in the DICOM standard dictionary. Be aware that un-selecting this may allow patient information residing in the private tags to persist into the anonymized data.

Note that Patient information burnt into images (such as DRRs) will NOT be removed.

DICOM Information Display

Information from the DICOM standard may be displayed while viewing DICOM instances by selecting Tools|Display DICOM Info. With this option displayed, hovering the mouse over an attribute will show the allowable VM range, VR type and length, IOD and module name, tag type and description.

If you need more information, the DICOM standard along with the IEC standard are also available under Help|DICOM Standards.

IHE-RO Information Display

Information from the IHE-RO Transactions standard may be displayed separately or in conjunction with DICOM information by selecting Tools|Display IHE-RO Info. IHE-RO only applies to CTs, Structure Sets, RT Plans and Dose objects and only to a select few attributes defined for those objects.

For more information regarding IHE-RO, the IHE-RO Integration Profiles and Transaction documents are available under Help|DICOM Standards.

Add/Remove and Copy/Paste Attributes

Attributes may be added or removed at the study, series or instance level. Right click the tag view and select Add or Remove to insert or remove an attribute. The remove dialog will give you the option of removing all matching attributes within the study, series or instance or just that attribute highlighted.

At the instance level, attributes may be copied and pasted between instances by right-clicking the tag(s) and selecting Copy or via the shortcut Ctrl+c. To paste attributes on the clipboard, highlighting the sequence or neighboring attributes you would like to paste in or near, right-clicking and selecting Paste or by way of the shortcut Ctrl+v.

Find Attributes

To search for an attribute in a study, series or instance, right-click the attribute view and select Find Attribute . . . . Any matching attribute will be highlighted when found.

Raw Pixel Sampling

To sample pixel values from an image or dose, select the Image View tab when displaying the file and click the point in the image you wish to sample. The selected pixel will be highlighted in green and the raw values will be displayed along with the location of the pixel in the image.

Validation

DICOM and IHE-RO validation is available under Tools|DICOM Validate and Tools|IHE-RO Validate, respectively.

Validation results are displayed in-view. Existing attributes out of compliance with the DICOM standard will be highlighted in the attribute view. Red highlighting pertains to errors, yellow highlighting to warnings and grey to internal errors and information. When an attribute has various types of messages associated with it, errors will trump all other messages and warnings will trump internal errors/information. Instances in the overview view will contain messages regarding missing required attributes. If an instance contains errors, the [symbol] icon will be displayed next to it. If a file contains warnings but no errors, the [symbol] icon will appear and if a file only contains information messages, the [symbol] will appear. If the instance contains no issues and is in compliance, the [symbol] will be displayed. Series will display the state of its most severe instance. Studies will display the state of its most severe series.

Details regarding the nature of validation failure for attributes and instances can be accessed via tooltip by hovering over the attribute or instance.

DICOM Validation

DICOM validation checks existing attributes within each file against the DICOM standard to verify such properties as value representation and multiplicity are respected as well as required attributes exist when they should.

A natural language parser is employed to interpret conditional attributes and is not perfect. As a result, the validator makes use of the ExplicitConditionalValidations.config configuration file which allows users to add existence criteria for Type 1C and 2C attributes. For each attribute listed in this file, a flag may be set to tell the validator to either ignore the file or use a list of user-provided conditions to verify existence of an attribute when the DICOM condition description is too complicated for the parser.

IHE-RO Validation

Because IHE-RO is an extension of the DICOM standard, the IHE-RO validator first runs DICOM validation before validating against the IHE-RO standard. The IHE-RO validator checks value perpetuation of a handful of attributes from CT series down to Dose. It will also perform a series of attribute value and existence checks in addition to those checks done by the DICOM validator. While IHE-RO only defines modules for RT Plan and Dose IODs, the IHE-RO validator will validate CTs and Structure Sets as well as RT Plans and Doses against any matching attribute it can find in the IHE-RO modules.

DICOM Repository Queries

To search the DICOM repository a user must first ensure that he/she can connect to \\10.137.115.124 via the network. CarpeDICOM will attempt to make this connection on starting up and if it is unable to do so the File|Search Database . . . item will be disabled. If it is enabled, you are connected to this server.

Select File|Search Database . . . to bring up the search dialog or right-click a group of highlighted attributes you wish to base your search on and select Search Database . . . .

Simple Queries:

For simple queries, enter the group and element number along a value you wish to search on. If no value is entered, the search will look for all files with that attribute regardless of its value. Press search to initiate the search.

Advanced Queries:

Select the Advanced tab and enter your SQL query into the text box. Hit Search to initiate the search.

For the database schema, please contact Ryan Renne.

Note that In order to load data from an advanced query, ensure you include the “PatientID” and “FilePath” columns found in the DCMRecord table.

To load a file, highlight the file and hit “Load File”. Only that file will be loaded. To load the dataset containing a file matching your query, highlight a file and hit “Load Patient” to load that file and all other files associated with its Patient ID.

Files loaded will be copied locally to the hidden directory .dbTemp in the write location specified in Edit|Preferences . . . .

Note that Loading data will cause CarpeDICOM to copy it locally across the network, a task that may take a while for overseas users.

Comparing DICOM Files (CarpeCompare)

To compare attributes between two files, select the first file you wish to compare against in CarpeDICOM. Then select Tools|Compare DICOM . . . to bring up the DICOM comparison dialog. The file path for the other file being compared must then be entered into the DICOM File 2 text box. Hit compare to view the differences between files.

Rows will display in red if the value of an attribute between both files differs. If one file contains the attribute but the other does not, that attribute will display grey.

Images and doses are visually diffed in the Images or Doses tabs, respectively. The left-most image is the difference image. Two images or doses with a different number of rows, columns, transfer syntaxes or bits allocated will not be diffed. Matching pixels between images or doses will appear pink, and close matches will appear white. The more different the pixel, the closer to black the pixel will appear.

CarpeCompare works as a standalone tool as well and does not have to be accessed through CarpeDICOM.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, an apparatus, system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include software code, such as microcode, assembly language code, a higher-level language code, or the like. Such software code can include computer readable instructions for performing various methods. The software code may form portions of computer program products or computer program modules. Further, in an example, the software code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method for creating one or more test objects used for radiation therapy having a processor executing a plurality of computer executable instructions, said instructions comprising:
defining a plurality of parameters to create a test object;
determining a shape of an object to be used for imaging purposes;
selecting an image modality;
generating a plurality of images based on the test objects based on object parameters, shape, and image modality;
creating contours on the plurality of test images;
generating a radiation therapy treatment plan, which defines a plurality of radiation beams to be applied to a target located on the test images;
creating a radiation therapy dose to be applied to the target; and
generating a plurality of Digital Imaging and Communication (DICOM) files that represents one or more radiation therapy objects for a particular test anatomy.

2. The method of claim 1, wherein an image modality comprises at least one of a CT, a MRI, a PET, and an X-ray modality.

3. The method of claim 1, wherein determining a shape further comprises determining a size and a location of the object.

4. The method of claim 1, wherein the plurality of parameters comprises at least a type of anatomy, a pixel value for an image, a pixel value for a radiation dose, a center of the object, and a radius of the object.

5. The method of claim 1, wherein the radiation therapy treatment plan is a barchtherapy treatment plan.

6. The method of claim 1, wherein the radiation therapy treatment plan is for an ion treatment plan.

7. The method of claim 1, wherein the radiation therapy treatment plan is for at least one of a linear accelerator, an integrated MRI-Linear accelerator, a gamma knife, and an integrated MRI-gamma knife.

8. The method of claim 1, further comprising creating a digitally reconstructed radiograph (DRR) based on the radiation therapy treatment plan

9. The method of claim 1, wherein creating contours comprises creating a structure set.
